(19) United States
(12) Patent Application Publication
(10) Pub. No.: US 2025/0236665 A1
(43) Pub. Date: Jul. 24, 2025

(54) USTEKINUMAB DOSAGE FORMS AND METHODS

(71) Applicant: Rani Therapeutics, LLC, San Jose, CA (US)

(72) Inventors: Mir IMRAN, Los Altos Hills, CA (US); Mir HASHIM, San Jose, CA (US)

(73) Assignee: Rani Therapeutics, LLC, San Jose, CA (US)

(21) Appl. No.: 19/175,798

(22) Filed: Apr. 10, 2025

Related U.S. Application Data
(63) Continuation of application No. PCT/US2023/034100, filed on Sep. 29, 2023.
(60) Provisional application No. 63/416,854, filed on Oct. 17, 2022.

(51) Int. Cl. C07K 16/24; A61K 9/00; A61K 9/48; A61K 39/00

(52) U.S. Cl. CPC C07K 16/244; A61K 9/0092; A61K 9/4808; A61K 9/4858; A61K 2039/505; A61K 2039/542; A61K 2039/545; C07K 2317/21; C07K 2317/31; C07K 2317/76

(57) ABSTRACT

The present disclosure relates generally to ingestible devices containing anti-interleukin antibodies that bind to one or both of human interleukin-23 and human interleukin-12, such as ustekinumab, wherein the devices are structured and formulated to deliver therapeutically effective amounts of the antibodies into a gastrointestinal (GI) lumen wall or into and through a GI lumen wall into a peritoneum or peritoneal cavity after ingestion of the device, and achieve desired pharmacokinetic and therapeutic effects.

### CROSS-REFERENCE TO RELATED APPLICATION

This application is a continuation of International Patent Application No. PCT/US2023/034100, filed Sep. 29, 2023, and claims the benefit of and priority to U.S. Provisional Application No. 63/416,854, filed Oct. 17, 2022, the entire contents of which are hereby incorporated by reference.

### FIELD OF INVENTION

The present disclosure relates generally to compositions, dosage forms, devices, and methods for delivery of anti-interleukin antibodies such as ustekinumab by ingestion. More specifically, the present disclosure relates to ingestible dosage forms (e.g., ingestible devices) that contain a composition comprising anti-interleukin antibodies such as ustekinumab that are structured and formulated to deliver therapeutically effective amounts of anti-interleukin antibody into a gastrointestinal (GI) lumen wall (e.g., stomach wall, intestinal wall, colon, etc.) of the subject or into and through a GI lumen wall into a peritoneum or peritoneal cavity of the subject (and thereafter into the bloodstream) to achieve desired pharmacokinetic and therapeutic results. The compositions, devices, and methods may be useful, for example, for the treatment of psoriasis (including moderate to severe plaque psoriasis), psoriatic arthritis, Crohn's disease (including moderate to severe Crohn's disease), ulcerative colitis (including moderate to severe ulcerative colitis), and other conditions in which treatment with an anti-interleukin antibody may be indicated.

### BACKGROUND

The following discussion is merely provided to aid the reader in understanding the disclosure and is not admitted to describe or constitute prior art thereto.

The anti-interleukin antibody product STELARA® (ustekinumab) currently is approved for use in the U.S. for the treatment of moderate to severe plaque psoriasis, psoriatic arthritis, moderate to severe Crohn's disease, and moderate to severe ulcerative colitis. STELARA® is administered by subcutaneous or intravenous injection. The dosing regimen for patients weighing up to 100 kg is 45 mg administered subcutaneously initially and 4 weeks later, followed by 45 mg administered subcutaneously every 12 weeks. The dosing regimen for patients weighing over 100 kg is 90 mg administered subcutaneously initially and 4 weeks later, followed by 90 mg administered subcutaneously every 12 weeks.

Shaped masses comprising biologically active proteins or polypeptides, including anti-interleukin antibodies, have been described, for example, in U.S. Pat. Nos. 10,098,931 and 10,227,403. Other anti-interleukin antibody preparations are described in U.S. Pre-Grant Publication 2018/0251537.

There remains a need for compositions, dosage forms, devices, and methods for delivery of anti-interleukin antibodies such as ustekinumab by ingestion.

### SUMMARY

In one embodiment, a method of administering anti-interleukin antibodies to a subject in need thereof is provided. The method includes administering to the subject by ingestion an ingestible device containing a payload formed from, or containing, a composition comprising anti-interleukin antibodies. The device is structured to deliver the composition into a gastrointestinal (GI) lumen wall of the subject or into and through a GI lumen wall into a peritoneum or peritoneal cavity of the subject. The anti-interleukin antibodies bind to one or both of human interleukin-23 and human interleukin-12.

In another embodiment, an ingestible device for use in delivering anti-interleukin antibodies to a subject in need thereof is provided. The device contains a payload formed from, or containing, a composition comprising anti-interleukin antibodies. The device is structured to deliver the composition into a gastrointestinal (GI) lumen wall of the subject or into and through a GI lumen wall into a peritoneum or peritoneal cavity of the subject. The anti-interleukin antibodies bind to one or both of human interleukin-23 and human interleukin-12.

In one or more embodiments, the anti-interleukin antibodies are antagonists of one or both of human interleukin-23 and human interleukin-12.

In one or more embodiments, the anti-interleukin antibodies comprise ustekinumab or a biosimilar thereof.

In one or more embodiments, the anti-interleukin antibodies comprise ustekinumab.

In one or more embodiments, the anti-interleukin antibodies exhibit a bioavailability that is greater than a bioavailability of subcutaneously administered anti-interleukin antibodies.

In one or more embodiments, the anti-interleukin antibodies exhibit a bioavailability that is substantially the same as a bioavailability of subcutaneously administered anti-interleukin antibodies.

In one or more embodiments, the subject is suffering from one or more conditions selected from psoriasis, moderate to severe plaque psoriasis, psoriatic arthritis, Crohn's disease, and ulcerative colitis.

In one or more embodiments, the device contains a dose of anti-interleukin antibodies of from about 0.25 mg to about 0.75 mg. In one or more embodiments, the device contains a dose of anti-interleukin antibodies selected from about 0.5 mg and about 0.75 mg. In one or more embodiments, the device contains a dose of anti-interleukin antibodies of from about 3.75 to about 15 mg.

In one or more embodiments, the device is administered once daily.

In one or more embodiments, the composition is a solid composition that comprises the anti-interleukin antibodies, a buffering agent, a surfactant, and an antioxidant. In one or more embodiments, the composition (e.g., the solid composition) comprises the anti-interleukin antibodies, L-histidine/L-histidine hydrochloride, polyoxyethylene sorbitan monooleate, and L-methionine. In one or more embodiments, the composition (e.g., the solid composition) further comprises a lubricant and a bulking agent. In one or more embodiments, the composition (e.g., the solid composition) comprises the anti-interleukin antibodies, L-histidine/L-histidine hydrochloride, polyoxyethylene sorbitan monooleate, trehalose, PEG 3350, and L-methionine. In one or more embodiments, the composition (e.g., the solid composition) comprises about 67% w/w ustekinumab, about 10% w/w L-histidine/L-histidine HCl, about 1% w/w polyoxyethylene sorbitan monooleate, about 10% w/w trehalose, about 10% w/w PEG 3350, and about 2% w/w L-methionine. In one or more embodiments, the composition (e.g., the solid composition) comprises about 75% w/w ustekinumab, about 12% w/w L-histidine/L-histidine HCl, about 1% w/w polyoxyethylene sorbitan monooleate, about 3% w/w trehalose, about 7% w/w PEG 3350, and about 2% w/w L-methionine.

In one or more embodiments, the composition is a liquid composition.

In one or more embodiments, the payload is in the form of a solid tissue penetrating member structured to be inserted into a GI lumen wall of the subject and/or be inserted into a peritoneum or peritoneal cavity of the subject after ingestion of the device. In one or more embodiments, the composition is filled in a hollow, biodegradable microneedle constituting the solid tissue penetrating member. In one or more embodiments, the composition is a shaped mass constituting the solid tissue penetrating member. In one or more embodiments, the device comprises multiple payloads of the anti-interleukin antibodies. In one or more embodiments, the device is structured to deliver one or more payloads into the GI lumen wall, peritoneum, or peritoneal cavity of the subject at different times. In one or more embodiments, the GI lumen wall is a wall of the stomach.

In one or more embodiments, the GI lumen wall is a wall of the intestine. In one or more embodiments, the wall of the intestine is a wall of the small intestine.

In one or more embodiments, the ingestible device is comprised within a swallowable capsule.

In one or more embodiments, the method or use comprises an induction dosing period following by a maintenance dosing period.

In one or more embodiments, the subject is an adult patient weighing ≤100 kg or a child patient weighing >60 kg, and
the induction dosing period comprises an induction dosing regimen selected from:
   a 45 mg subcutaneous dose at week 0 and week 4;
   once daily administration of about 1.5 mg by the ingestible device for a period of time on the order of 4 weeks;
   once daily administration of about 12 mg by the ingestible device for a period of time on the order of 4 weeks;
   once daily administration of about 9 mg/day by the ingestible device for 7 days beginning at week 0 and again at week 4; and
the maintenance dosing period comprises a maintenance dosing regimen selected from:
   a 45 mg subcutaneous dose every 12 weeks;
   once daily administration of about 0.5 mg by the ingestible device;
   once weekly administration of about 3.75 mg by the ingestible device;
   a once monthly short course of daily administration of about 2.5 mg/day by the ingestible device for 6 days;
   once quarterly administration of about 15 mg by the ingestible device;
   a once quarterly short course of daily administration of about 12 mg/day by the ingestible device for 5 days;
wherein the amounts refer to the dose of anti-interleukin antibody, optionally wherein the anti-interleukin antibody is ustekinumab or a biosimilar thereof,
wherein the method or use comprises administration of at least one dose of anti-interleukin antibody by the ingestible device.

In one or more embodiments, the subject is an adult patient weighing >100 kg, and
the induction dosing period comprises an induction dosing regimen selected from:
   a 90 mg subcutaneous dose at week 0 and week 4;
   once daily administration of about 3.25 mg by the ingestible device for a period of time on the order of 4 weeks;
   twice daily administration of about 1.5 mg by the ingestible device for a period of time on the order of 4 weeks;
   once daily administration of about 24 mg by the ingestible device for a period of time on the order of 4 weeks;
   once daily administration of about 18 mg/day by the ingestible device for 7 days beginning at week 0 and again at week 4; and
the maintenance dosing period comprises a maintenance dosing regimen selected from:
   a 90 mg subcutaneous dose every 12 weeks;
   once daily administration of about 1 mg by the ingestible device;
   twice daily administration of about 0.5 mg by the ingestible device;
   once weekly administration of about 7.5 mg by the ingestible device;
   a once monthly short course of twice daily administration of about 2.5 mg by the ingestible device for 6 days;
   once quarterly administration of about 30 mg by the ingestible device;
   a once quarterly short course of daily administration of about 12 mg/day by the ingestible device for 9 days;
wherein the amounts refer to the dose of anti-interleukin antibody, optionally wherein the anti-interleukin antibody is ustekinumab or a biosimilar thereof,
wherein the method or use comprises administration of at least one dose of anti-interleukin antibody by the ingestible device.

### BRIEF DESCRIPTION OF THE DRAWINGS

FIG. 1A (FIG. 1A) shows an example of an embodiment of an expandable component of a self-sizing device as disclosed herein prior to being folded and/or rolled, and an example of an embodiment of a capsule as disclosed herein.

FIG. 1B (FIG. 1B) shows the expandable component of FIG. 1A in an embodiment of a folded and/or rolled arrangement prior to disposing the expandable component in the capsule.

FIG. 1C (FIG. 1C) shows the expandable component of FIG. 1A in the folded and/or rolled arrangement of FIG. 1B and disposed within the capsule.

FIG. 2A (FIG. 2A), FIG. 2B (FIG. 2B), and FIG. 2C (FIG. 2C) show an example of an embodiment of a capsule as disclosed herein including a self-sizing device as disclosed herein, including an expandable component within a degradable capsule, as the device traverses a lumen.

FIG. 2D (FIG. 2D), FIG. 2E (FIG. 2E), and FIG. 2F (FIG. 2F) show a progression of the expandable component of FIG. 2C in a rotated view as the expandable component expands within the lumen.

FIG. 3 (FIG. 3) shows an example of an embodiment of a self-sizing device as disclosed herein including an expandable component in a fully-extended state within a lumen.

FIG. 4A (FIG. 4A) shows an example of an embodiment of a self-sizing device as disclosed herein including an expandable component with one non-compliant section and no hinge.

FIG. 4B (FIG. 4B) shows an example of an embodiment of a self-sizing device including an expandable component with two non-compliant sections and one hinge.

FIG. 5 (FIG. 5) shows an embodiment of a capsule as disclosed herein encompassing an ingestible device. Inset A shows a fully assembled enteric-coated capsule. Schematic B shows various parts and components of the capsule.

FIG. 6 (FIG. 6) shows results of a computer-based pharmacokinetic model simulation of ustekinumab dosing based on published human pharmacokinetic data, plotting simulated plasma concentration vs. time curves from a pharmacokinetic model simulation of ustekinumab dosing at 45 mg once every 12 weeks (top curve) vs. daily dosing of 0.75 mg (middle curve) or 0.5 mg (bottom curve). The dashed line reflects a minimum therapeutic plasma level of 0.69 μg/mL.

FIG. 7 (FIG. 7) shows the plasma concentration vs. time curve obtained after oral administration of a single dose of an ingestible dosage form of ustekinumab as described herein in the canine study of Example 2.

FIG. 8 (FIG. 8) shows the plasma concentration vs. time curves obtained after administration of a single dose of ustekinumab via an ingestible device or single subcutaneous (SC) injection in the canine study of Example 4.

### DETAILED DESCRIPTION

The present disclosure provides compositions, dosage forms, devices, and methods for delivery of anti-interleukin antibodies such as ustekinumab using an ingestible dosage form (an ingestible device) containing a payload formed from, or containing, a composition including the antibodies, where the device is structured to deliver the anti-interleukin antibodies into a GI lumen wall (e.g., stomach wall, intestinal wall, colon, etc.) of the subject or into and through a GI lumen wall into a peritoneum or peritoneal cavity of the subject (and thereafter into the bloodstream). The payload may be in the form of a solid tissue penetrating member structured to penetrate a GI lumen wall of the subject and/or be inserted into a peritoneum or peritoneal cavity of the subject after ingestion of the device. The ingestible device may be disposed within a capsule, such as a swallowable capsule.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more”.

As used herein, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the terms “substantially” and “about” when used with a numerical value mean the numerical value stated as well as up to and including plus or minus 10% of the numerical value. For example, “about 10” should be understood as both “10” and “in a range between and including 9 and 11”.

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “comprising”, “comprise”, “comprises”, “includes”, and “including” are intended to mean that the compositions and methods include the recited elements, but do not exclude others.

As used herein, the phrase “therapeutically effective amount” with reference to an anti-interleukin antibody such as ustekinumab means a dose that provides the specific pharmacological effect for which the drug is administered in a subject in need of such treatment. It is emphasized that a therapeutically effective amount of anti-interleukin antibody will not always be effective in treating the condition for which it is administered in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, the condition being treated, and/or the severity of the condition of the subject being treated.

A “therapeutic level” of circulating anti-interleukin antibody in a human subject with reference to an anti-interleukin antibody such as ustekinumab means a plasma level associated with the specific pharmacological effect for which the drug is administered in a subject in need of such treatment. A “therapeutic level” of circulating ustekinumab in a human subject may be a plasma concentration of about 0.69 μg/mL or greater. That is, a plasma concentration of ustekinumab of about 0.69 μg/mL or greater may be therapeutically effective.

The term “ingest” or a grammatical variation thereof (e.g., “ingesting”, “ingestion,” “ingested” or “ingestible”) refers herein to taking into the stomach, whether by swallowing or by other means of depositing into the stomach (e.g., by depositing into the stomach by endoscope or depositing into the stomach via a port).

The term “lumen” refers herein to the inside space of a tubular structure. Examples of lumens in a body include arteries, veins, and tubular cavities within organs. The term “gastrointestinal lumen” or “GI lumen” refers generally to any lumen of the GI tract (e.g., a lumen of the esophagus, stomach, small intestine, large intestine, or colon); the term “GI lumen wall” refers to a lumen wall of a GI lumen.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” as used herein refers herein to the intake/expulsion system of a body including, for example, the mouth, pharynx, esophagus, stomach, pylorus, small intestine, cecum, large intestine, colon, rectum, anus, and valves or sphincters therebetween.

The terms “individual,” “subject,” and “patient” are used interchangeably herein, and include any individual mammalian subject (e.g., bovine, canine, feline, equine, or human). In specific embodiments, the subject, individual, or patient is a human.

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “manufacture” or a grammatical variation thereof (e.g., “manufacturing” or “manufactured”) as related to a component, device, composition, or system refers herein to making or assembling the component, device, composition, or system. Manufacture may be wholly or in part by hand and/or wholly or in part in an automated fashion.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking.

The term “non-degradable” refers to an expectation that degradation will be minimal, or within a certain acceptable design percentage, for at least an expected duration in an expected environment.

The terms “anti-interleukin antibody composition” and “composition of anti-interleukin antibodies” may be used interchangeably herein and refer to a composition including one or more components where at least one of the components is an anti-interleukin antibody. The anti-interleukin antibody composition can include, for example, anti-interleukin antibodies either as the sole active agent or including one or more additional active agents.

The term “anti-interleukin antibodies” as used herein refers to a full-length antibodies and fragments thereof that bind to an interleukin protein, such as one or both of human interleukin-23 and human interleukin-12, and includes monoclonal antibodies, human antibodies, and humanized antibodies. An antibody may be an immunoglobulin (Ig) molecule comprised of four polypeptide chains: two heavy (H) chains and two light (L) chains. A functional antibody fragment may comprise a fragment of an antibody, such as at least one complementarity determining region (CDR) of a heavy chain, at least one complementarity determining region (CDR) of a light chain, a heavy chain variable region, a light chain variable region, a heavy chain constant region, a light chain constant region, a framework region, etc. Anti-interleukin antibodies as discussed herein may bind to one or both of human interleukin-23 and human interleukin-12. Anti-interleukin antibodies as discussed herein may be antagonists of one or both of human interleukin-23 and human interleukin-12.

The anti-interleukin antibodies may be or comprise ustekinumab, or antibodies biosimilar thereto. Ustekinumab (present in the FDA-approved product STELARA®) is a recombinant human IgG1κ monoclonal antibody produced in a murine cell line (Sp2/0) that acts as a human interleukin-12 and human interleukin-23 antagonist. Ustekinumab is comprised of 1326 amino acids and has an estimated molecular mass that ranges from 148,079 to 149,690 Daltons.

As used herein, a “delay agent” refers to a component included in the composition to slow a release rate of one or more other component(s) from a composition. A delay agent may be, for example, poly(lactic acid) (PLA), poly(glycolic acid) (PGA), polyethylene glycol (PEG), poly(ethylene oxide) (PEO), poly (l-lactic acid) (PLLA), poly(D-lactic acid) (PDLA), another polymer, or a hydrogel.

Various abbreviations may be used herein for standard units, such as deciliter (dl), milliliter (ml), microliter (μl), international unit (IU), centimeter (cm), millimeter (mm), nanometer (nm), inch (in), kilogram (kg), gram (gm), milligram (mg), microgram (μg), nanogram (ng), millimole (mM), degrees Celsius (° C.), degrees Fahrenheit (° F.), millitorr (mTorr), hour (hr), minute (min), second (s or sec), millisecond (ms), microsecond (ps), or nanosecond (ns).

Disclosed herein are ingestible dosage forms (ingestible devices) useful for delivering anti-interleukin antibodies to a subject in need thereof. The devices contain a payload formed from, or containing, a composition comprising anti-interleukin antibodies, and are structured to deliver the composition into a GI lumen wall of the subject or into and through a GI lumen wall into, e.g., a peritoneum or peritoneal cavity of the subject, after ingestion. As noted above, the anti-interleukin antibodies provided in the devices may bind to one or both of human interleukin-23 and human interleukin-12 and/or may be antagonists of one or both of human interleukin-23 and human interleukin-12. As noted above, the anti-interleukin antibodies provided in the devices may be or comprise ustekinumab or a biosimilar thereof. Although the anti-interleukin antibodies provided in the devices are not limited to ustekinumab, for the sake of convenience the discussion that follows refers to ustekinumab. It should be understood that other anti-interleukin antibodies could be used in addition to or instead of ustekinumab, including antibodies that are biosimilar to ustekinumab, and/or other antibodies that bind to one or both of human interleukin-23 and human interleukin-12 and/or may be antagonists of one or both of human interleukin-23 and human interleukin-12.

### II. Ingestible Dosage Forms (Ingestible Devices)

As noted above, the ingestible dosage forms described herein are ingestible devices that contain a payload made from or comprising or containing a composition including ustekinumab. The payload may be in the form of a solid tissue penetrating member structured to penetrate a GI lumen wall of the subject and be inserted into the GI lumen wall or surrounding bodily environment (e.g., peritoneum or peritoneal cavity) of the subject after ingestion of the device (e.g., after swallowing the device or after ingestion of the device by another mode, such as via an endoscope or stomach port), thereby delivering the ustekinumab into the blood stream of the subject. The payload may be formed from, or may contain, the composition. Specific embodiments of such devices are described in more detail below. Any devices disclosed or referenced herein optionally may be disposed within a capsule, such as a swallowable capsule. Typically, the materials used to form the components of the devices are classified as food grade, food additive, active or inactive food ingredient or GRAS (Generally Recognized As Safe by the FDA). The devices (including any capsule shell) may be biodegradable.

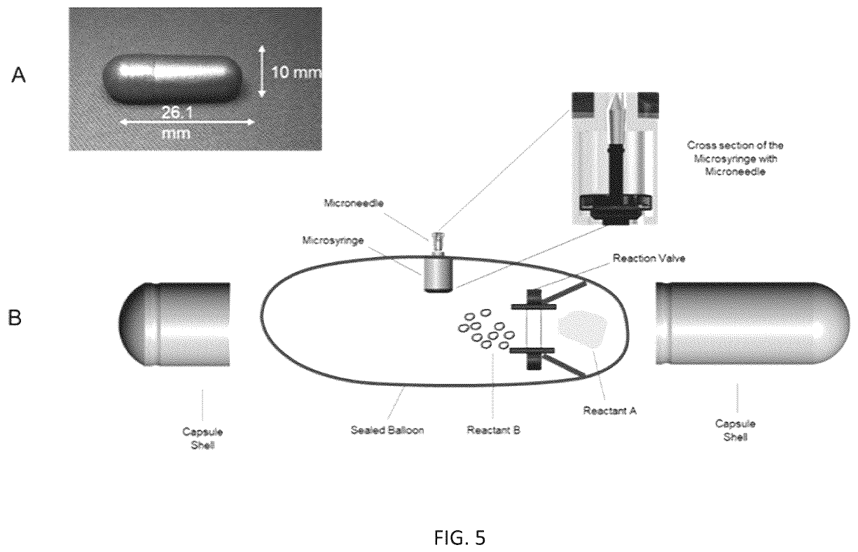

FIG. 5 diagrammatically illustrates an ingestible device (e.g., a swallowable device) according to an embodiment of this disclosure. As illustrated, the device is disposed within a capsule, which may be any suitably sized swallowable capsule, such as a 000 sized HPMC (hydroxypropyl methylcellulose) capsule, or such as the capsule illustrated in inset A of FIG. 5. The capsule may be provided with a coating, such as an enteric coating, such as an enteric polymer, (e.g., a polymer that dissolves at a target pH within the digestive tract). For example, the enteric coating can be selected to dissolve at a pH>6 to avoid dissolution in the acidic environment of the stomach. In embodiments in which the device is incorporated into an enteric coated capsule, after the capsule leaves the stomach and enters the duodenum, the higher intestinal pH of the small intestine dissolves the enteric coating and capsule shell, exposing an interior of the device and the components contained therein to intestinal fluid.

Thus, in some embodiments, the capsule and/or the coating are designed to degrade in the presence of a particular chemical, or after a certain length of time under the conditions expected at a target site within the GI tract (e.g., under certain values or ranges of pH, temperature, pressure exerted against the capsule, or a combination of the foregoing). In an embodiment, the coating includes a material to improve swallowability and/or ingestibility, such as guar gum or xanthan gum.

The device may contain a mechanism to effectuate delivery of the payload into a location of the GI tract, such as the intestinal wall or into and through an intestinal wall into, e.g., a peritoneum or peritoneal cavity of the subject. In an embodiment, the mechanism includes an actuator that is triggered when the device is at a target delivery site, such as within the intestine. In other embodiments, the mechanism may effectuate delivery of the payload at another location in the GI tract, such as the stomach, large intestine, or colon.

In the embodiment of FIG. 5, the device contains a sealed polyethylene balloon (labeled Sealed Balloon), a delivery construct (labeled Microsyringe), and an actuator in the form of two reactants and a valve, all of which together constitute a mechanism to effectuate delivery of the payload into the intestinal wall or into and through an intestinal wall into, e.g., a peritoneum or peritoneal cavity of the subject. In this embodiment, the Microsyringe contains the payload (labeled Microneedle). The two reactants, labeled Reactant A and Reactant B (e.g., citric acid and potassium bicarbonate), are separated by a valve (labeled Reaction Valve) in an initial state. When exposed to fluid such as intestinal fluid (e.g., after the capsule degrades), a portion of the valve degrades and allows the two reactants to mix, which results in formation of a gas (e.g., carbon dioxide or other innocuous gas) that expands the balloon. Inflation of the balloon aligns the Microsyringe substantially perpendicular to the long axis of the intestine and builds pressure in the balloon sufficient to provide the force needed to eject the Microneedle from the Microsyringe and inject the Microneedle into the intestinal wall or surrounding tissue thereof. After microneedle deployment, the balloon deflates and the balloon and other components are excreted out through the GI tract with normal bowel movements.

In general, the mechanism to effectuate delivery of the payload into the intestinal wall may include various mechanical, electrical, electromechanical, and/or chemical components. For example, springs, levers, and/or various movable components may together constitute the mechanism.

The payload may be disposed within a protective enclosure until just before (e.g., in terms of ms or ps) the payload is delivered into the intestinal wall. For example, in the embodiment of FIG. 5, the Microneedle may be sealed within the Microsyringe in the initial state, and when ejected from the Microsyringe into the intestinal wall, may pierce or break a seal on the Microsyringe to pass out of the Microsyringe.

After entering the intestinal wall or surrounding bodily environment (e.g., peritoneum or peritoneal cavity), the payload dissolves or degrades in the moist tissue environment to release the ustekinumab composition into the blood stream.

The payload may be a hollow structure in which the ustekinumab composition is disposed, or the payload may itself be formed from the ustekinumab composition (e.g., the ustekinumab composition may be compressed into a desired size and shape). In an embodiment, the ustekinumab composition is compressed into a cylindrically-shaped microtablet or other shaped microtablet. In an embodiment, the ustekinumab composition (in uncompressed or compressed form) is disposed within a degradable shell, and the shell containing the microtablet is the payload. In an embodiment, the shell is approximately needle-shaped. Degradation of the payload may occur within 1, 2, 3, 4, or 5 minutes of injection into the intestinal wall, or may take longer. Properties of the payload (e.g., structure and/or material) may be selected to delay release of the ustekinumab from the payload.

The device may include a detectable marker, such as a radiolabel, to assist in tracking the device as it proceeds through the subject's GI tract. Examples of markers include, but are not limited to, barium sulfate (whose dispersion indicates fluid ingress inside the device and imminent delivery of the payload) and bismuth, which optionally may be included on or in a component contained in the device to radiographically track the component's transit along the GI tract as well as to confirm its excretion.

In an embodiment, which may be similar to the embodiment illustrated in FIG. 5, a balloon is self-sizing. With respect to embodiments including the self-sizing balloon, the balloon is referred to herein as an “expandable component”. Such embodiments are described in more detail in the following paragraphs.

In some embodiments, the expandable component includes multiple sections. In the expanded configuration of the expandable component, at least one of the sections is non-compliant and at least one of the sections is compliant. Compliant refers to a state in which the section may be readily deformed, and non-compliant refers to a state in which the section resists deformation. Absent a constraint, each section will expand to a fully-extended state and the expandable component will reach its maximum dimensions, which may depend, for example, on materials used to form the expandable component and/or a capacity of an expansion module that is implemented (e.g., for expansion by inflation, maximum dimensions may be influenced by a limitation on an inflation force available from the expansion module, or by a stretch factor of a material or materials of the expandable component). The expansion module is part of a mechanism to effectuate delivery of the payload into the intestinal wall.

Each compliant section operates in a similar fashion to a hinge and thus will be referred to for convenience as a hinge. In a fully-extended state of the expandable component (where each section is fully extended), each hinge has at least one design dimension (width, length, and/or circumference) that is substantially smaller than a corresponding design dimension of each non-compliant section.

As the expandable component expands, it will achieve a shape that reflects an equilibrium between a force exerted by the expandable component on an interior of a lumen of the GI tract and a force of the lumen against the expandable component. Said another way, the expandable component may not fully extend and will tend to be bent about the hinge if constrained by the lumen. This bending is due to the smaller dimension(s) of the hinge as compared to the corresponding dimension(s) of the non-compliant section(s), which in the expanded configuration of the expandable component leads to lower rigidity of the hinge as compared to rigidity of the non-compliant section(s).

In this way, the expandable component expands until the non-compliant sections (which may hereinafter be referred to as NCS) are pressed against the lumen inner wall to maintain the expandable component in a position suitable for delivering a ustekinumab composition to the lumen wall, for a time at least sufficient to accomplish such delivery. The expandable component may then be deflated.

The expandable component may be removed after deflation, may be left to degrade in situ, or may be allowed to pass out of the lumen. The expandable component can be structured from degradable materials such that the expandable component degrades after a designed period of time after exposure to an environment at a target site.

A self-sizing device as described above may be used to deliver a therapeutic ustekinumab composition into a GI lumen wall (e.g., stomach wall, intestinal wall, colon) via ingestion (e.g., swallowing or other mode of ingestion). The self-sizing device may include a capsule. The self-sizing device may include a coating on the capsule and/or on the expandable component (e.g., a coating as described above), designed to degrade at a particular location within the GI tract, such as within the intestine. In an embodiment, all or portions of the capsule and/or the coating are designed to degrade in the presence of a particular chemical, or after a certain length of time under the conditions expected at a target site within the GI tract (e.g., under certain values or ranges of pH, temperature, pressure exerted against the capsule, or a combination of the foregoing). In an embodiment, the coating includes a material to improve swallowability and/or ingestibility, such as guar gum or xanthan gum.

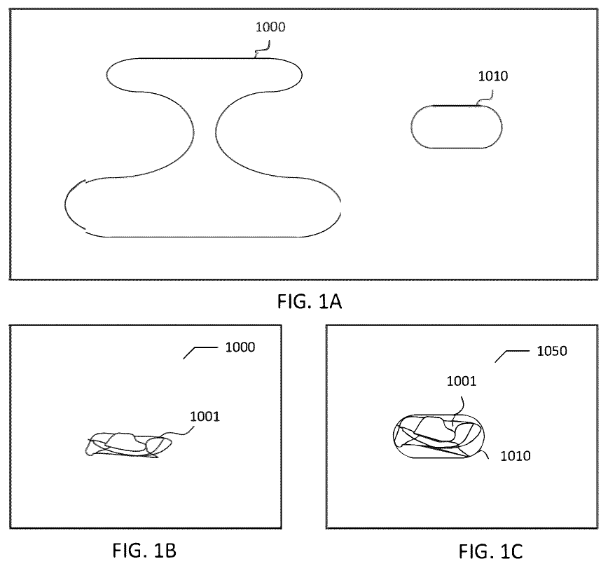

FIGS. 1A-1C illustrate an example of assembling a self-sizing device 1050, which is an ingestible device as described herein. FIG. 1A illustrates an embodiment of an expandable component 1000 and an embodiment of a capsule shell 1010. Outer dimensions of expandable component 1000 may be several times larger than inner dimensions of capsule 1000. For example, a longest dimension of an embodiment of expandable component 1000 may be two to five times greater than an inner length of capsule shell 1010. Other ratios of outer dimensions of an expandable component to inner dimensions of a capsule are within the scope of the present disclosure. FIG. 1B illustrates expandable component 1000 folded and/or rolled into an arrangement 1001 that is smaller than inner dimensions of capsule shell 1010. As illustrated, arrangement 1001 is folded and/or rolled in a complex arrangement in this embodiment, origami style. FIG. 1C illustrates arrangement 1001 disposed into capsule 1010, and capsule shell 1010 assembled together. Device 1050 includes expandable component 1000 (in arrangement 1001) and capsule shell 1010, and may include other components not shown such as an expansion module, a delivery component, one or more coatings, and a deflation valve.

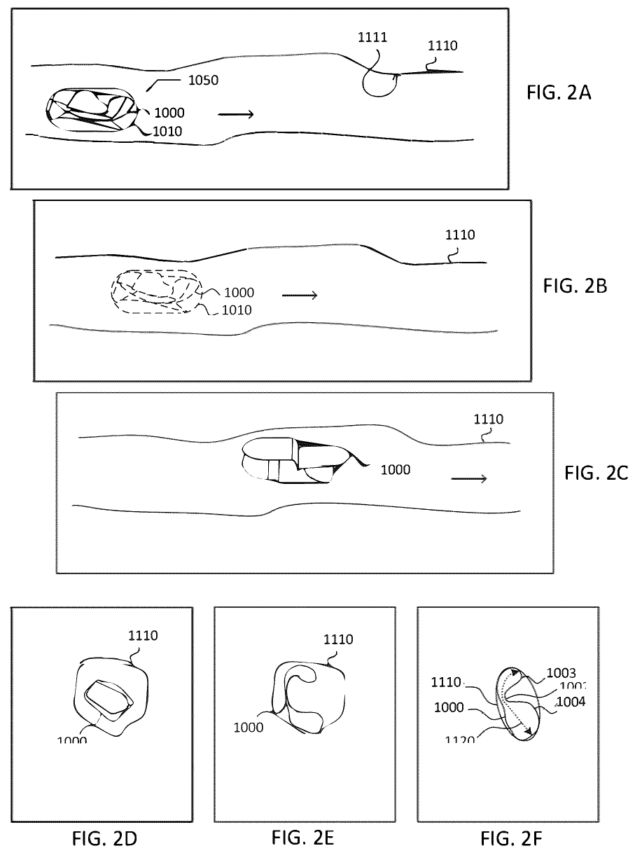

FIGS. 2A-2F illustrate an example of how device 1050 may be provided to a target delivery site in the GI tract and position expandable component 1000 to deliver a therapeutic ustekinumab composition into a lumen wall 1111 of a GI lumen 1110 (e.g., an intestinal wall, stomach wall, etc.). Referring back to FIGS. 2A-2C, device 1050 is shown from a view approximately perpendicular to a direction of travel of device 1050 (indicated by an arrow), whereas device 1050 in FIGS. 2D-2F is shown as if looking into lumen 1110 (e.g., approximately 90 degrees from the view of FIGS. 2A-2C).

FIG. 2A illustrates device 1050 within lumen 1110 shown in a relaxed state (e.g., between peristaltic contractions). In this embodiment, an outer diameter of device 1050 is less than an inner diameter of lumen 1110 at this point in the GI tract. Device 1050 traverses lumen 1110, for example through operation of peristalsis, fluid dynamics, and/or gravity.

FIG. 2B illustrates that, after reaching the designed target site or after encountering conditions (e.g., pH) representative of the designed target site, capsule shell 1010 degrades (or is triggered to break open or degrade) as device 1050 continues to traverse lumen 1110. For example, with respect to delivery within the intestine, a target site may be in a general region (e.g., small intestine), a more specific region (e.g., jejunum), or a specific tissue area (e.g., a tissue area that has been marked).

FIG. 2C illustrates that, subsequent to (or concurrent with) capsule shell 1010 degrading, expandable component 1000 begins to unfurl from its folded and/or rolled arrangement.

FIG. 2D illustrates expandable component 1000 mostly unfurled, and FIG. 2E illustrates expandable component 1110 as it is expanded (e.g., by an expansion module).

FIG. 2F illustrates expandable component 1000 after expansion. In this embodiment, expandable component 1000 would have been able to expand if not constrained by lumen 1110 until a reference line 1120 was approximately a straight line. However, lumen 1110 exerts force against expandable component 1000 which causes expandable component 1000 to bend at a hinge 1002. An NCS 1003 and an NCS 1004 of expandable component 1000 then press against the lumen wall of lumen 1110 (e.g., at opposing sides of the lumen) and position expandable component 1000 to deliver a therapeutic ustekinumab composition at or into the lumen wall. Hinge 1002 may also press against the lumen wall. In an embodiment, peristalsis may move expandable component 1000 in its expanded state through lumen 1110. In an embodiment, expandable component 1000 may have sufficient internal pressure (combined with surface tension) to firmly hold expandable component 1000 in approximately a same position even during peristalsis. In an embodiment, expandable component 1000 includes a surface roughness or surface protrusions to assist in maintaining a position of expandable component 1000.

Expansion of an expandable component may be accomplished by any suitable expansion module. In an embodiment, a spring mechanism is released from a compressed state to deploy, for example, a filler piece of firm or soft material to push outwards and thus expand the expandable component. In an embodiment, two or more reactants are mixed together to form a gas and thus expand the expandable component. In an embodiment, a material is rapidly combusted to generate a gas and thus expand the expandable component. Other expansion modules are within the scope of the present disclosure.

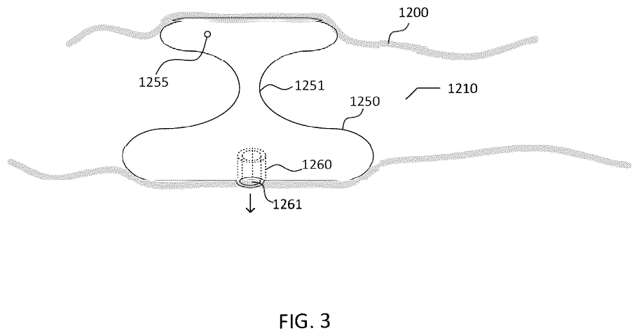

FIG. 3 illustrates a lumen 1200 in which a self-sizing device 1210 including an expandable component 1250 is positioned. In this embodiment, the force of lumen 1200 against expandable component 1250 is not sufficient to substantially cause a hinge 1251 of expandable component 1250 to bend, so expandable component 1250 is in an approximately fully-extended state. Said another way, a bending of hinge 1251 of expandable component 1250 is negligible at this position in this lumen 1200.

Expandable component 1250 may be expanded by generation of gas, and may include a valve 1255 to release the gas from within expandable component 1250 into lumen 1200 to deflate expandable component 1250. Valve 1255 may include one or more components. In an embodiment, valve 1255 is a pinch valve that degrades in the presence of fluid (e.g., biological matter) to reveal a port or an opening defined by expandable component 1250. In an embodiment, valve 1255 is a piece of material or a coating over a port or an opening in expandable component 1250, and the piece of material or coating is designed to degrade a period of time after expandable component 1250 delivers a therapeutic ustekinumab composition. In an embodiment, valve 1255 is opened manually.

The therapeutic composition of ustekinumab (or multiple therapeutic compositions) can be stored within expandable component 1250 in any constitution (e.g., as a solid, fluid, slurry, or powder). The therapeutic composition(s) can be delivered in any constitution (e.g., as a solid, fluid, slurry, or powder). In some embodiments, two or more components of the therapeutic composition are mixed within expandable component 1250 prior to delivery. In an embodiment, expandable component 1250 includes a portal such as portal 1260 defining an opening 1261 through which the therapeutic composition is delivered. In embodiments that include a tissue penetrating member, the tissue penetrating member may be delivered through the portal.

The therapeutic ustekinumab composition is actively released such that the therapeutic ustekinumab composition is forcibly expelled from expandable component 1250 and into lumen 1200 or beyond the lumen wall into the surrounding bodily environment (e.g., through the mucosa, submucosa, muscularis, serosa, outer wall of lumen 1200, and into the peritoneal cavity); in such an embodiment, the therapeutic ustekinumab composition may be released through one or more portals such as portal 1260.

The therapeutic composition of ustekinumab may be forcibly expelled from expandable component 1250 by a delivery mechanism. For example, with respect to a solid composition (e.g., tablet, pellet, or pointed form), the therapeutic ustekinumab composition may be expelled by way of a spring mechanism that is released to quickly eject the solid composition out of expandable component 1250, or by way of a piston mechanism in which the piston is moved by a spring mechanism, or a gas expansion to push the solid composition quickly out of expandable component 1250.

With respect to a fluidic composition (e.g., a liquid), the therapeutic ustekinumab composition may be expelled as a fluid jet by way of a spring mechanism or gas acting on a piston or plunger to eject the preparation from a reservoir through an opening or nozzle of expandable component 1250. The fluid jet of the therapeutic ustekinumab composition can have sufficient force to actively penetrate the GI lumen wall and enter the blood stream.

Additionally or alternatively, the fluidic composition including ustekinumab may be expelled through a hollow needle coupled to expandable component 1250. The hollow needle can penetrate into and/or through the GI lumen wall by way of a spring mechanism or gas acting on a piston or plunger to eject the composition through the needle and into the GI lumen wall or surrounding tissue thereof.

In some embodiments, a surface of expandable component 1250 through which the therapeutic ustekinumab composition is delivered contacts the wall of lumen 1200 along a stretch of approximately 5 mm to 20 mm.

In some embodiments, the therapeutic ustekinumab composition is delivered from expandable component 1250 from multiple surfaces. Such embodiments may include more than one tissue penetrating member that is formed from or contains a ustekinumab composition.

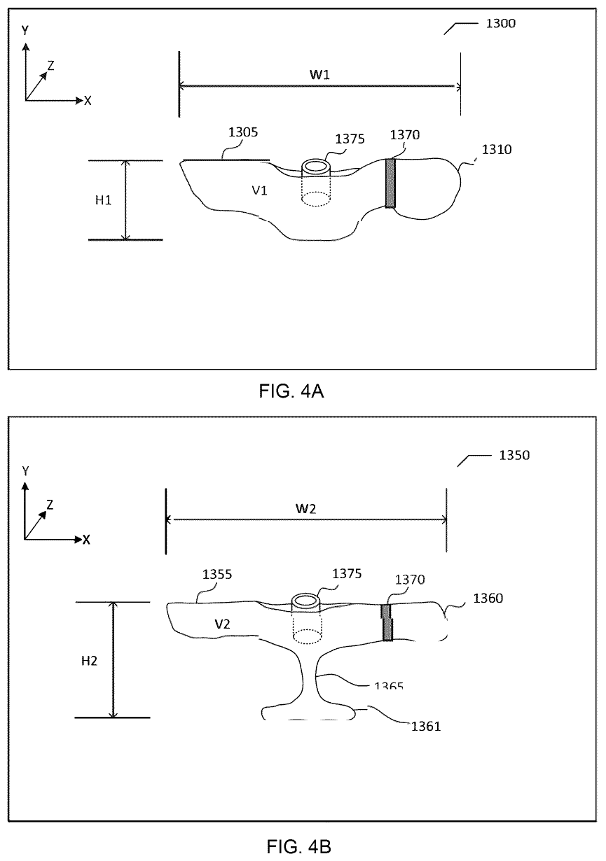

FIG. 4A illustrates an embodiment of a self-sizing device 1300 including an expandable component 1305 which includes one NCS 1310 and no hinge. FIG. 4B illustrates an embodiment of a self-sizing device 1350 including an expandable component 1355 which includes two NCSs, an NCS 1360 and an NCS 1361, and a hinge 1365 between. With respect to these embodiments, a width W1 of expandable component 1305 is similar to a width W2 of expandable component 1355, whereas a height H1 of expandable component 1305 is less than a height H2 of expandable component 1355.

Multiple devices 1300 (FIG. 4A) structured according to a same design will have approximately a same maximum circumference of NCS 1310 (in a y-z plane) when fully inflated in a lumen due to the non-conformance of NCS 1310, such that expandable component 1305 does not significantly adapt to a variety of different internal lumen circumferences. Different sizes of device 1300 may therefore be appropriate for different animal species and/or different subjects within a species. In contrast, multiple devices 1350 (FIG. 4B) structured according to a same design will likely not have a same maximum circumference (in a y-z plane) when fully inflated in a lumen because each device 1350 will adjust to the size of the lumen in which it is inflated due to the flexibility (non-conformance, bending) of hinge 1365 even when inflated. Maximum circumference with respect to device 1350 within the lumen refers to a maximum circumference of the shape that device 1350 takes within the lumen (e.g., a partially-extended shape or a fully-extended shape).

Specific materials can be chosen to confer desired structural and material properties to the payload (e.g., column strength for insertion into the lumen wall, or porosity and/or hydrophilicity for controlling disintegration of the payload and thus the release of a therapeutic ustekinumab composition). For example, certain materials may be more suitable when the payload is formed as a tissue penetrating member, whereas other materials may be more suitable when the payload is contained within a tissue penetrating member. In an embodiment, a tip of the payload includes or is coated with a degradable material such as sucrose, maltose, or other sugar, to increase hardness and tissue piercing properties of the tip. Once positioned within a lumen wall, the payload may be degraded by interstitial fluids within tissue so that the therapeutic ustekinumab composition dissolves and is absorbed into the blood stream. Properties of a payload such as size, shape, and chemical composition can be selected to allow for dissolution and absorption of a drug in a matter of seconds, minutes, or hours. Rates of dissolution can be controlled through various excipients such as disintegrants (e.g., starch, sodium starch glycolate, or a cross-linked polymer such as carboxymethyl cellulose). The choice of disintegrants may be specifically adjusted for the environment within a lumen wall (e.g., blood flow and average number of peristaltic contractions).

A payload can be fabricated entirely from a ustekinumab composition, or can define a cavity that includes a ustekinumab composition. A self-sizing device can include and deliver one or more payloads, each of which can contain the same or a different ustekinumab composition. Each payload can have different properties; for example, two payloads may be designed to deliver respective compositions concurrently (e.g., two instances of one composition, or one instance each of two distinct compositions), or may be designed to deliver respective compositions at different times (e.g., to provide a subsequent dose of a same ustekinumab composition or to provide a different ustekinumab composition subsequently).

### III. Microtablets

Various embodiments of microtablets comprising a ustekinumab composition in the form of solid shaped masses comprising ustekinumab and methods for forming such solid shaped masses are described. A shaped mass may comprise a therapeutic composition that comprises a drug (e.g., ustekinumab) and one or more pharmaceutically acceptable excipients, including degradable material (such as a sugar, polyethylene, lactic acid polymer, poly(glutamic acid-co-lactic acid-co-glycolic acid (PGLG), etc.) which degrades within a target delivery site in the body (e.g., the wall of the small intestine or peritoneum or peritoneal cavity) to release drug.

The shaped mass can be formed from a variety of shaping processes known in the pharmaceutical arts. Typically, the shaped mass will be formed by a compression process such as compression molding. According to one or more embodiments, the biological activity of the ustekinumab in the shaped mass is at least about 70% of that prior to compression, at least 80% of that prior to compression, at least 85% of that prior to compression, about 90% of that prior to compression, or at least 95% of that prior to compression.

The shaped mass can have a density in a range from about 0.60 mg/mm³ to about 1.15 mg/mm³, from about 0.70 mg/mm³ to about 1.15 mg/mm³, from about 0.80 mg/mm³ to about 1.10 mg/mm³, from about 1.02 to 1.06 mg/mm³, or from about 1.03 to 1.05 mg/mm³.

The shape of the shaped mass may be any shape suitable for use as a component of a device as described herein, e.g., any shape suitable for disposing within a tissue-penetrating member (e.g., a pellet or cylindrical shape) or any shape suitable for use as a tissue-penetrating member (e.g., a needle or dart shape). The shaped mass may comprise a pellet shape, or may have a tablet, conical, pyramidal, hot dog/capsule like, arrow head, cylindrical, cube, sphere, hemisphere or other suitable shape. For embodiments of the shaped mass having a cylindrical or pellet shape, the shaped mass may have a diameter in the range from about 0.5 mm to 1 mm and a length from about 1.75 mm to 3.25 mm.

As noted above, according to various embodiments, the shaped mass can be formed in part from a degradable material that is configured to dissolve or otherwise degrade, e.g., in the wall of the intestine or in the peritoneum or peritoneal cavity, so as to release the drug for systemic absorption. Suitable degradable materials include various sugars such as trehalose, maltose and sucrose, various lactic acids polymers such as polyglycolic acid (PGA), polylactic acid (PLA); polyglycolic lactic acid (PGLA); poly(glutamic acid-co-lactic acid-co-glycolic acid (PGLG); various polyethylenes such as high density, low density and linear low density PE and PEO (polyethylene oxide), various cellulose polymers such as HPMC (hydroxypropyl methyl cellulose), CMC (carboxy methyl cellulose), MC (methyl cellulose), methacrylic acid-ethyl acrylate copolymer, methacrylic acid-methyl methacrylate copolymer PVOH (polyvinyl alcohol), silicone rubber. and other biodegradable polymers known in the art. The material and other properties of the degradable polymer and shaped mass can be selected to produce selectable rates of degradation in the intestinal wall or other target delivery site (e.g., peritoneum or peritoneal cavity).

A description will now be provided of a fabrication process used to make various embodiments of a drug-containing shaped mass as described herein. The process includes a process for fabricating a powder containing the drug, and a shaped mass formation process for forming the powder into shaped masses. For convenience, the shaped masses are referred to as microtablets; however it should be appreciated that other forms and/or shapes are equally applicable, as described above.

A process for formulation of a powder (e.g., dry composition) comprising the drug (e.g., ustekinumab) will now be described. A typical process may include preparing a composition comprising the drug and excipients. The composition may be prepared by combining dry ingredients, or by preparing a solution comprising one or more of the ingredients, and then evaporating the solvent(s) (optionally comprising lyophilisation) to obtain a dry composition, and, optionally, adding one or more dry ingredients to obtain a composition comprising the drug and other excipients.

The drug (e.g., ustekinumab) may be present in the dry composition in any amount suitable for providing the target dose in the ingestible device, and in one or more embodiments, may be present in an amount of from about 1% to about 99% w/w of the composition, including from about 25% to about 75% w/w, including from about 50% to about 60% w/w, or from about 50% to about 80% w/w, including about 55% w/w, such as about 55.6% w/w, about 67% w/w, or about 75% w/w. In some embodiments, the drug (e.g., ustekinumab) is present in an amount of about 67% w/w, such as 67% w/w. In some embodiments, the drug (e.g., ustekinumab) is present in an amount of about 75% w/w, such as 75% w/w.

The excipients can include one or more of a buffering agent, an antioxidant, a surfactant, a lubricant, a binder, a stabilizing agent, and a bulking agent.

The buffering agent may be a histidine HCl buffer system (e.g., L-histidine and L-histidine monohydrochloride monohydrate (L-histidine/L-histidine HCl)), and in one or more embodiments, may be present in an amount of from about 1% to about 15% w/w of the composition, including from about 1% to about 5% w/w or from about 2% to about 3% w/w, including about 2.4% w/w, or from about 10% to about 15% w/w, including about 10% w/w or about 12% w/w of, e.g., L-histidine/L-histidine HCl. In some embodiments, the buffering agent (e.g., L-histidine/L-histidine HCl) is present in an amount of about 10% w/w, such as 10% w/w. In some embodiments, the buffering agent (e.g., L-histidine/L-histidine HCl) is present in an amount of about 12% w/w, such as 12% w/w.

The antioxidant may comprise L-methionine, and in one or more embodiments, may be present in an amount of from about 1% to about 10% w/w of the composition, including from about 3% to about 8% w/w, including about 5% w/w, such as about 4.4% w/w, or from about 1% to about 5% w/w, including from about 1% to about 3% w/w, including about 2% w/w. In some embodiments, the antioxidant (e.g., L-methionine) is present in an amount of about 2% w/w, such as 2% w/w.

The surfactant may be Polysorbate 80 (polyoxyethylene sorbitan monooleate, such as Tween 80) and in one or more embodiments, may be present in an amount of from about 0.05% to about 3% w/w of the composition, including from about 0.05% to about 1% w/w, or from about 1% to about 3% w/w, including about 0.1% w/w and about 1% w/w. In some embodiments, the surfactant (e.g., polyoxyethylene sorbitan monooleate) is present in an amount of about 1% w/w, such a 1% w/w.

The lubricant may be used to facilitate both microtablet formation and ejection from a mold. The lubricant may comprise polyethylene glycol 3350 (PEG 3350) and in one or more embodiments, may be present in an amount of from about 5% to about 50% w/w or from about 10% to about 50% w/w of the composition, including from about 5% to about 15% w/w and from about 20% to about 30% w/w, including about 7% w/w, about 10% w/w, or about 25% w/w. In some embodiments, the lubricant (e.g., PEG 3350) is present in an amount of about 10% w/w, such as 10% w/w. In some embodiments, the lubricant (e.g., PEG 3350) is present in an amount of about 7% w/w, such as 7% w/w.

The bulking agent or stabilizing agent may be trehalose, and in one or more embodiments, may be present in an amount of from about 1% to about 20% w/w or from about 5% to about 20% w/w of the composition, including from about 1% to about 5% w/w or from about 10% to about 15% w/w, including about 3% w/w or about 10% w/w, including about 12.5% w/w. In some embodiments, the bulking or stabilizing agent (e.g., trehalose) is present in an amount of about 10% w/w, such as 10% w/w. In some embodiments, the bulking or stabilizing agent (e.g., trehalose) is present in an amount of about 3% w/w, such as 3% w/w.

Alternatively, the bulking agent may be mannitol, and in one or more embodiments may be present in an amount of from about 5% to about 20% w/w of the composition, including from about 10% to about 15% w/w, including about 12.5% w/w. Other excipients may be added, such as one or more other binders, fillers, disintegrates, stabilizers, buffers and antimicrobials. For example, the composition may include sucrose, which may act as a stabilizer. If included, sucrose may be used in any suitable amount, such as from about 0.5% to about 4% w/w of the composition, including about 1.1% w/w of the composition, with the amount(s) of one or more other excipients adjusted accordingly.

In some embodiments the ustekinumab composition comprises about 55.6% w/w ustekinumab, about 2.4% w/w L-histidine/L-histidine HCl, about 0.1% w/w polyoxyethylene sorbitan monooleate, about 4.4% w/w L-methionine, about 25% w/w PEG 3350, and about 12.5% w/w trehalose. In some embodiments the ustekinumab composition comprises about 55.6% w/w ustekinumab, about 2.4% w/w L-histidine/L-histidine HCl, about 0.1% w/w polyoxyethylene sorbitan monooleate, about 4.4% w/w L-methionine, about 25% w/w PEG 3350, and about 12.5% w/w mannitol.

In some embodiments a ustekinumab composition as described herein comprises about 67% w/w ustekinumab, about 10% w/w L-histidine/L-histidine HCl, about 1% w/w polyoxyethylene sorbitan monooleate, about 10% w/w trehalose, about 10% w/w PEG 3350, and about 2% w/w L-methionine. In some embodiments a ustekinumab composition as described herein comprises about 75% w/w ustekinumab, about 12% w/w L-histidine/L-histidine HCl, about 1% w/w polyoxyethylene sorbitan monooleate, about 3% w/w trehalose, about 7% w/w PEG 3350, and about 2% w/w L-methionine.

The selection and proportions of the different ingredients may be taken into consideration during the formulation process so as to achieve a desired therapeutic dose of the drug with desired properties including desired tabletting properties, and desired release properties from the resulting microtablet.

The composition obtained as described above may be milled to produce a fine powder. For example, the composition may be placed in a low-protein-binding tube along with a single high-density milling ball, such as one made of stainless steel or yttrium-stabilized zirconium. The milling may be done using a rotator at max speed containing the tube film-wrapped to avoid moisture absorption or contamination. An ice pack may be placed on top of the tube to keep it cold. The room temperature can be controlled in a range for example from 60 to 64° F. The size of the milling tube, mass of the milling ball and duration of mixing may be selected to produce particular powder grain sizes, grain size homogeneity and powder density. For example, for the production of a 40 mg to 100 mg batch capacity, the use of a bottom-rounded 2 mL tube, a milling ball having a 0.44 g mass and a milling duration of 3 hours have resulted in fine and consistent grain sizes, achieving homogeneous and reliable density values.

The microtablet formation process is typically done via compression using a compression mold or other fixture to apply a compressive force to the fine powder including the drug (e.g., ustekinumab). A semiautomatic or fully automatic compression fixture may be used, as described in U.S. Pat. No. 10,098,931, the entire contents of which are incorporated herein by reference. As noted above, in various embodiments, the shaped mass can be formed into the shape of a tissue-penetrating member, i.e. it can be formed to have a tissue-penetrating shape by using molding or other like methods. In these and related embodiments, the shaped mass can have a needle or a dart-like shape (with or without barbs) configured to penetrate and/or be retained in to a tissue, or a membrane such as the intestinal wall.

After the microtablet is fabricated, the length, weight, density and bioactivity of the drug in the pellet may be measured. The bioactivity of the drug in the microtablet may be assayed using an enzyme-linked immunosorbant assay (ELISA) or other immune assay known in the art.

As described above, the shaped mass can be in the form of a tissue-penetrating member (e.g. in the form of a dart or needle) or contained in a tissue-penetrating member that is configured to be advanced into an intestinal wall or into and through an intestinal wall into a peritoneum or peritoneal cavity and biodegrade to release the ustekinumab in the intestinal wall, peritoneum or or peritoneal cavity and then the bloodstream.

### IV. Methods of Treatment and Pharmaceutical Uses

Also provided herein are treatments (methods of treatment and pharmaceutical uses) comprising administering an ingestible device according to any embodiments described herein by ingestion to a subject in need thereof, e.g., by the subject swallowing the device (orally administering) or by ingesting the device by another mode, such as via an endoscope or stomach port. In some embodiments, the treatments (methods of treatment and pharmaceutical uses) comprise once daily administration of an ingestible device as described herein. In some embodiments, the treatments (methods of treatment and pharmaceutical uses) comprise alternative dosing regimens as described in more detail below.

In general, the disclosed methods of treatment and pharmaceutical uses are contemplated for treating conditions such as psoriasis (including moderate to severe plaque psoriasis), psoriatic arthritis, Crohn's disease (including moderate to severe Crohn's disease), ulcerative colitis (including moderate to severe ulcerative colitis), and other conditions in which treatment with an anti-interleukin antibody may be indicated. In specific embodiments, the disclosed methods of treatment and pharmaceutical uses are for treating plaque psoriasis. In specific embodiments, the disclosed methods of treatment and pharmaceutical uses are for treating psoriatic arthritis. In specific embodiments, the disclosed methods of treatment and pharmaceutical uses are for treating Crohn's disease. In specific embodiments, the disclosed methods of treatment and pharmaceutical uses are for treating ulcerative colitis.

The disclosed methods of treatment and pharmaceutical uses may be used for any subject in need of administration of, e.g., ustekinumab, including human and non-human mammals. In human subjects, the age and size of the human is not limited, and may include pediatric, adult, and geriatric subjects, as well as subjects at, above, or below a normal range of body weight or height.

In some embodiments, the ingestible device or a payload of the ingestible device contains a dose of ustekinumab effective to achieve a therapeutic plasma level of ustekinumab for the condition being treated. In some embodiments, the ingestible device or a payload of the ingestible device contains a dose of ustekinumab effective to achieve a plasma level of ustekinumab of about 0.69 μg/mL or greater, including a plasma level of ustekinumab of 0.69 μg/mL or greater.

In some embodiments, the ingestible device or a payload of the ingestible device contains a dose of ustekinumab of about 0.5 mg, or more. In some embodiments, the ingestible device or a payload of the ingestible device contains a dose of ustekinumab of about 0.5 mg. In some embodiments, the ingestible device or a payload of the ingestible device contains a dose of ustekinumab of about 0.75 mg. In some embodiments, the ingestible device or a payload of the ingestible device contains a dose of ustekinumab of about 10 mg. In some embodiments, the ingestible device or a payload of the ingestible device contains a dose of ustekinumab of about 20 mg.

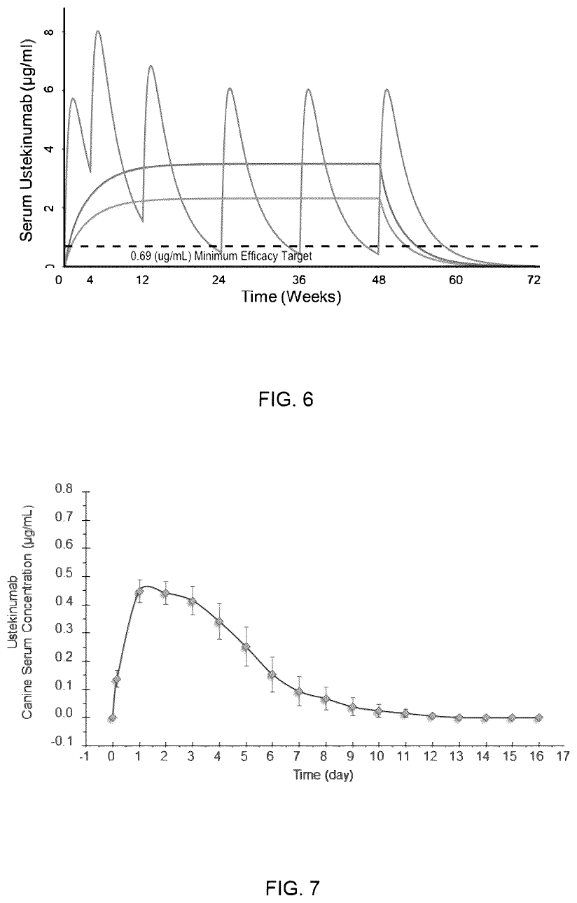

As reflected in FIG. 6, a computer-based pharmacokinetic model simulation of ustekinumab dosing based on published human pharmacokinetic data indicates that daily dosing of 0.5 mg will achieve plasma concentrations of 0.69 μg/mL or greater, e.g., will achieve plasma concentrations greater than 0.69 μg/mL (see FIG. 6, bottom curve; dashed line=0.69 μg/mL). The simulated data also indicate that daily dosing will maintain steady state therapeutic serum concentrations more effectively than the once-every-12-weeks subcutaneous regimen. Thus, in some embodiments, the ingestible device or a payload of the ingestible device contains a dose of ustekinumab less than 0.5 mg, such as 0.45 mg, 0.4 mg, 0.35 mg, 0.3 mg, or 0.25 mg.

In some embodiments the payload of the ingestible device contains a solid therapeutic composition comprising ustekinumab, and one or more other excipients, such as a microtablet composition as described above. In some embodiments the payload of the ingestible device contains an ustekinumab composition comprising ustekinumab, L-histidine/L-histidine HCl, polyoxyethylene sorbitan monooleate, and L-methionine, as described above, optionally further including PEG 3350, and about trehalose and/or mannitol. In some embodiments the payload of the ingestible device contains a solid ustekinumab composition comprising about 67% w/w ustekinumab, about 10% w/w L-histidine/L-histidine HCl, about 1% w/w polyoxyethylene sorbitan monooleate, about 10% w/w trehalose, about 10% w/w PEG 3350, and about 2% w/w L-methionine. In some embodiments the payload of the ingestible device contains a solid ustekinumab composition comprising about 75% w/w ustekinumab, about 12% w/w L-histidine/L-histidine HCl, about 1% w/w polyoxyethylene sorbitan monooleate, about 3% w/w trehalose, about 7% w/w PEG 3350, and about 2% w/w L-methionine.

In other embodiments, the ingestible device or a payload of the ingestible device contains a liquid therapeutic composition comprising ustekinumab, and one or more other excipients.

For treatment of psoriasis and psoriatic arthritis in adult patients weighing ≤100 kg and in children weighing >60 kg, STELARA® may be administered at an induction dose of 45 mg at weeks 0 and 4, followed by maintenance doses of 45 mg every 12^{th} week thereafter. For treatment of psoriasis and psoriatic arthritis in patients weighing >100 kg, STELARA® may be administered at an induction dose of 90 mg at weeks 0 and 4, followed by maintenance doses of 90 mg every 12^{th} week thereafter. A method of treatment or pharmaceutical use as disclosed herein may comprise an induction dosing period (such as, for example, an induction dosing period on the order of about 4 weeks, such as 4 weeks or 30 days), followed by a maintenance dosing period. Various exemplary induction dosing regimens and maintenance dosing regimens are disclosed below. It should be understood that any induction dosing regimen can be used in combination with any maintenance dosing regimen, and that different maintenance dosing regiments may be used over time (e.g., switching from one maintenance regimen to another). It also should be understood that any maintenance dosing regimen using a device as described herein could be used following an induction dosing period using a subcutaneously injected product, such as STELARA® or a biosimilar thereof. Likewise, any induction dosing regimen using a device as described herein, could be followed by a maintenance dose of a subcutaneously injected product, such as STELARA® or a biosimilar thereof. Further, any maintenance dose using a device as described herein could be followed by a maintenance dose of a subcutaneously injected product, such as STELARA® or a biosimilar thereof, or vice versa.

Sample induction dosing regimens for treating an adult patient weighing ≤100 kg or a child patient weighing >60 kg (amounts refer to dose of ustekinumab or a biosimilar thereof):
45 mg subcutaneous dose at week 0 and week 4
once daily administration of about 1.5 mg (including 1.5 mg) by a device as disclosed herein for a period of time on the order of 4 weeks (e.g., 4 weeks or 30 days)
once daily administration of about 12 mg (including 12 mg) by a device as disclosed herein for a period of time on the order of 4 weeks (e.g., 4 weeks or 30 days)
once daily administration of about 9 mg/day (including 9 mg/day) for 7 days beginning at week 0 and again at week 4

Sample maintenance dosing regimens for treating an adult patient weighing ≤100 kg or a child patient weighing >60 kg (amounts refer to dose of ustekinumab or a biosimilar thereof):
45 mg subcutaneous dose every 12 weeks
once daily administration of about 0.5 mg (including 0.5 mg) by a device as disclosed herein
once weekly administration of about 3.75 mg (including 3.75 mg) by a device as disclosed herein
once monthly short course of daily administration of about 2.5 mg/day (including 2.5 mg/day) by a device as disclosed herein for 6 days
once quarterly administration of about 15 mg (including 15 mg) by a device as disclosed herein
once quarterly short course of daily administration of about 12 mg/day (including 12 mg/day) by a device as disclosed herein for 5 days

Sample induction dosing regimens for treating an adult patient weighing >100 kg (amounts refer to dose of ustekinumab or a biosimilar thereof):
90 mg subcutaneous dose at week 0 and week 4
once daily administration of about 3.25 mg (including 3 mg or 3.25 mg) by a device as disclosed herein for a period of time on the order of 4 weeks (e.g., 4 weeks or 30 days)
twice daily administration of about 1.5 mg by a device as disclosed for a period of time on the order of 4 weeks (e.g., 4 weeks or 30 days)
once daily administration of about 24 mg (including 24 mg) by a device as disclosed herein for a period of time on the order of 4 weeks (e.g., 4 weeks or 30 days)
once daily administration of about 18 mg/day (including 18 mg/day) for 7 days beginning at week 0 and again at week 4

Sample maintenance dosing regimens for treating an adult patient weighing >100 kg (amounts refer to dose of ustekinumab or a biosimilar thereof):
90 mg subcutaneous dose every 12 weeks
once daily administration of about 1 mg (including 1 mg) by a device as disclosed herein
twice daily administration of about 0.5 mg (including 0.5 mg) by a device as disclosed herein
once weekly administration of about 7.5 mg (including 7.5 mg) by a device as disclosed herein
once monthly short course of twice daily administration of about 2.5 mg (including 2.5 mg) by a device as disclosed herein for 6 days
once quarterly administration of about 30 mg (including 30 mg) by a device as disclosed herein
once quarterly short course of daily administration of about 12 mg/day (including 12 mg/day) by a device as disclosed herein for 9 days

In some embodiments, an ingestible device as described herein containing a solid therapeutic composition of ustekinumab (or biosimilar thereof) is used to provide a dose of up to about 1 mg per device, such as a dose of about 0.5 mg or about 0.75 mg per device, including a dose of 0.5 mg or 0.75 mg per device, depending on the formulation of the composition and its concentration.

In some embodiments, an ingestible device as described herein comprising a liquid therapeutic composition of ustekinumab (or biosimilar thereof) is used to provide a dose of up to about 10 mg per device, such as for devices having a 100 μL capacity, or of up to about 20 mg per device, such as for devices having a 200 μL capacity, depending on the formulation of the composition and its concentration.

In some embodiments, dosing regimens described above using an ingestible device as described herein to provide a dose of up to about 3.25 mg (including 3 mg or 3.25 mg) contain a solid therapeutic composition of ustekinumab (or biosimilar thereof). In some embodiments, dosing regimens described above using an ingestible device as described herein to provide a dose of about 3.25 mg or greater (including 3 mg or greater or 3.25 mg or greater) contain a liquid therapeutic composition of ustekinumab (or biosimilar thereof).

In accordance with any embodiments using an ingestible device as described herein, a given dose can be provided by one ingestible device as described herein containing one or more payloads that collectively contain(s) the stated dose of ustekinumab (or biosimilar thereof), or by administering two or more ingestible devices as described herein, wherein the payload(s) of each device collectively contain(s) a portion of the stated dose of ustekinumab (or biosimilar thereof), e.g., by administration of one, two, three, 4, 5, 6, 7, 8, 9, 10, or more ingestible devices as described herein each comprising one, two, three, four or more payloads comprising a ustekinumab composition as described herein.

Currently approved anti-interleukin antibody products such as STELARA® (ustekinumab) require administration by subcutaneous or intravenous injection, which can be inconvenient and uncomfortable for patients. The present disclosure addresses this limitation by providing a route of administration that effectively delivers anti-interleukin antibodies into the bloodstream of a subject being treated without requiring injection, such as oral administration or ingestion by another mode, such as via an endoscope or stomach port. The disclosed treatments are more convenient and less invasive than currently approved anti-interleukin antibody products.

In addition to improved ease of administration, patient comfort, and patient compliance, administration of ustekinumab via an ingestible device as described herein may offer one or more other advantages, such as one or more of enhanced bioavailability and reduced immunogenicity as compared to another route of administration, such as subcutaneous injection. In some embodiments, ustekinumab administered via an ingestible device as described herein exhibits a bioavailability that is greater than a bioavailability of subcutaneously administered ustekinumab.

In some embodiments, ustekinumab administered via an ingestible device as described herein exhibits a bioavailability that is substantially the same as a bioavailability of subcutaneously administered ustekinumab.

Thus, in accordance with some aspects, the present disclosure provides methods of administering anti-interleukin antibodies (such as ustekinumab or a biosimilar thereof, or other anti-interleukin antibodies that bind to or are antagonists of one or both of human interleukin-23 and human interleukin-12) to a subject in need thereof, comprising administering to the subject by ingestion an ingestible device containing a payload formed from, or containing, a composition comprising the anti-interleukin antibodies, wherein the device is structured to deliver the composition into an intestinal wall of the subject or into and through an intestinal wall into a peritoneum or peritoneal cavity of the subject, after ingestion. As noted above, the administration may comprise oral administration (e.g., swallowing the device) or ingestion of the device by another mode, such as via an endoscope or stomach port. The payload may be in the form of a solid tissue penetrating member structured to penetrate an intestinal wall of the subject and be inserted into intestinal wall or into and through the intestinal wall into a peritoneum or peritoneal cavity of the subject after ingestion of the device. The administration of such ingestible devices may be repeated, for example, at a frequency of once every 1-3 days or less frequently, such as in accordance with any of the dosing regimens described above. The payload may be formed from, or may contain, the composition including anti-interleukin antibodies, such as a solid (dry) composition or liquid composition as described above. The payload is protected from the gastrointestinal (GI) tract environment (e.g., protected against degradation due to ingress of fluid reaching the payload) until the payload is injected into the wall of the small intestine, which is insensitive to sharp stimuli so that the subject does not register pain from the injection.

The present disclosure also provides methods of treating one or more conditions such as psoriasis (including moderate to severe plaque psoriasis), psoriatic arthritis, Crohn's disease (including moderate to severe Crohn's disease), ulcerative colitis (including moderate to severe ulcerative colitis), and other conditions in which treatment with an anti-interleukin antibody may be indicated, comprising administering by ingestion to a subject in need thereof an ingestible device as described herein, such as in accordance with any of the dosing regimens described above.

While specific embodiments have been described and illustrated, it should be understood that components or characteristics of one embodiment can be combined or substituted with one or more components or characteristics from other embodiments. Further, for any positive recitation of a component, characteristic, constituent, feature, step or the like, the present disclosure specifically contemplates the exclusion of that component, value, characteristic, constituent, feature, step or the like. It also should be understood that illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. Thus, the specification and drawings are to be regarded as illustrative rather than restrictive.

The following examples are given to illustrate the present invention. It should be understood, however, that the invention is not to be limited to the specific conditions or details described in these examples.

### EXAMPLES

### Example 1—Preparation of Ustekinumab Ingestible Devices (Formulation A)

Microtablets of ustekinumab were manufactured under aseptic conditions, targeting a dose of 0.5 mg per microtablet, with a target microtablet mass of about 0.9 mg.

A solution comprising ustekinumab, L-histidine/L-histidine HCl (as a buffering agent), polyoxyethylene sorbitan monooleate (as a surfactant), L-methionine (as an antioxidant), PEG 3350 (as a lubricant), and trehalose (as a bulking agent/stabilizing agent) was prepared and lyophilized to form the tableting composition. The tableting composition used to prepare the microtablets contained 55.6% w/w ustekinumab, 2.4% w/w L-histidine/L-histidine HCl (as a buffering agent), 0.1% w/w polyoxyethylene sorbitan monooleate (as a surfactant), 4.4% w/w L-methionine (as an antioxidant), 25% w/w PEG 3350 (as a lubricant), and 12.5% w/w trehalose (as a bulking agent/stabilizing agent). The microtablets were prepared using a compaction cylinder pressure of 10-90 PSI (e.g., 14-16 PSI) for an applied pressure on the tableting composition of 800-9000 PSI.

Ustekinumab activity was assessed in the ustekinumab solution, lyophile, and microtablet to ensure that the biological activity remained high and was not substantially degraded. Ustekinumab activity in the microtablet was greater than 80% of the target amount (0.5 mg) and greater than 80% of the amount used to prepare the microtablets.

The microtablets were incorporated into devices within a capsule as described above with reference to FIG. 5, with a payload in the form of a hollow microneedle containing a single microtablet, and the capsule device containing a single payload.

### Example 2—Orally Delivered Ustekinumab in Canines (Formulation A)

The ability to deliver ustekinumab in an ingestible device as described herein was assessed in canines. After an overnight fast, canines having a body weight of 8.5-11.5 kg were orally administered one capsule device as described in Example 1 above (n=7) at a dose of 0.5 mg. Fluoroscopic imaging was used to monitor capsule transit and confirm deployment within the small intestine. Blood levels of ustekinumab were assessed pre-dose, and at 4 hours, and 1, 2, 3, 4, 5, 6, 7, 8, 9, 10, 11, 12, 13, and 14 days post-administration. Results are reported in FIG. 7 and in the table below (mean±SEM).

| | Cmax (μg/mL) | Tmax (days) | AUC (μg · hr /mL) |
|---|---|---|---|
| Ingestible Device | 0.48 ± 0.05 | 2.0 ± 0.3 | 56 ± 10 |
| (n = 7) | | | |

The results showed that ustekinumab can be successfully delivered by an ingestible device as described herein, with a deployment success rate of 70%.

### Example 3—Preparation of Ustekinumab Ingestible Devices—(Formulations B and C)

Microtablets of ustekinumab were manufactured under aseptic conditions, targeting a dose of 0.5 mg or 0.75 mg per microtablet.

A solution comprising ustekinumab, L-histidine/L-histidine HCl (as a buffering agent), Tween 80 (as a surfactant), L-methionine (as an antioxidant), PEG 3350 (as a lubricant), and trehalose (as a stabilizing agent) was prepared and lyophilized to form tableting compositions as follows.

| | | Weight Percentage | |
|---|---|---|---|
| | | Formulation B | Formulation C |
| Component | Function | 0.5 mg Dose | 0.75 mg Dose |
| Ustekinumab | API | 67% | 75% |
| L-Histidine/L- | Buffer | 10% | 12% |
| Histidine-HCl | | | |
| Tween 80 | Surfactant | 1% | 1% |
| Trehalose | Stabilizer | 10% | 3% |
| PEG 3350 | Lubricant | 10% | 7% |
| L-Methionine | Antioxidant | 2% | 2% |

The 0.5 mg doses were prepared in 0.75 mg microtablets, while the 0.75 mg doses were prepared in 1 mg microtablets. Both types of microtablets were prepared using a compaction cylinder pressure of 10-90 PSI for an applied pressure on the tableting composition of 800-9000 PSI. Each microtablet was incorporated into a device within a capsule as described above with reference to FIG. 5, with a payload in the form of a hollow microneedle containing a single microtablet, and the capsule device containing a single payload.

### Example 4—Orally Delivered Ustekinumab in Canines (Formulations B and C)

The ability to deliver ustekinumab in an ingestible device as described herein was assessed in canines. The pharmacokinetic profile of a single dose of ustekinumab administered via an ingestible device as described herein was compared to the pharmacokinetic profile obtained through single subcutaneous (SC) injection. Canines having a body weight of 7.0-9.0 kg were divided into 3 groups as follow:
Group 1 (SC group; n=6): 0.5 mg/0.5 mL subcutaneous injection of ustekinumab;
Group 2 (ingestible device group; n=8): one capsule device containing microtablets containing 0.5 mg ustekinumab as described in Example 3; and
Group 3 (ingestible device group; n=8): one capsule device containing microtablets containing 0.75 mg ustekinumab as described in Example 3.

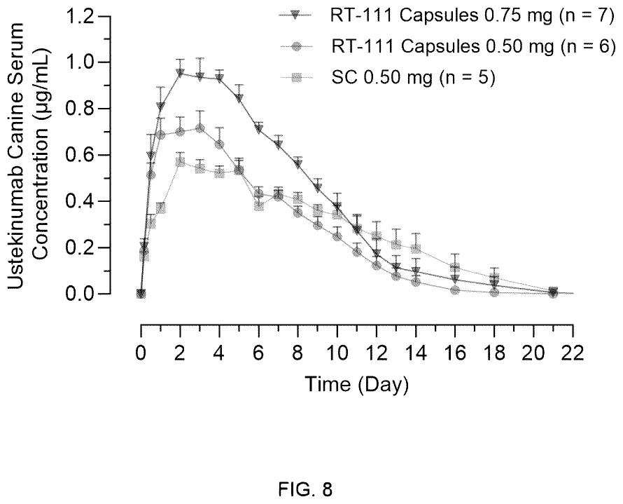

After fasting for at least 12 hours, canines were administered ustekinumab according to the study groups described above. Fluoroscopic imaging was used to monitor capsule transit and confirm deployment within the small intestine. Blood levels of ustekinumab were assessed pre-dose, and at 4 hours, 12 hours, and days 1-14, 16, 18, 21, 23 and 27 days post-administration. Results are reported in FIG. 8 and in the table below (mean±SEM).

| | Pharmacokinetic Parameters | | | |
|---|---|---|---|---|
| | (Mean ± SEM) | | | |
| Administration | Cmax | Tmax | AUC | AUC* |
| Route | (μg/mL) | (days) | (day · μg/mL) | ((day · μg/mL)/mg) |
| Ingestible Device | 1.05 ± 0.08 | 2 ± 0 | 8.23 ± 0.61 | 10.97 ± 0.81 |
| 0.75 mg (n = 7) | | | | |
| Ingestible Device | 0.75 ± 0.07 | 2 ± 0 | 5.63 ± 0.59 | 11.25 ± 1.19 |
| 0.50 mg (n = 6)** | | | | |
| SC | 0.60 ± 0.05 | 3 ± 1 | 6.01 ± 0.68 | 12.02 ± 1.37 |
| 0.50 mg (n = 5)*** | | | | |

*Dose-normalized
**Two animals suspected to have anti-drug antibodies (ADAs) were excluded from the data set
***One animal suspected to have ADAs and was excluded from the data set

The results show that ustekinumab can be successfully delivered by an ingestible device as described herein, with a pK success rate of 100% (for all animals in which the device successfully deployed). The devices containing microtablets with 0.75 mg and 0.5 mg ustekinumab showed 91%, and 94% relative bioavailability, respectively.

These data underscore the surprising advantages of the compositions, dosage forms, devices, and methods disclosed herein, especially when compared to the current standard of care that requires injections of ustekinumab.

### Example 5—Phase I Clinical Trial

A Phase 1 clinical study will be conducted in healthy volunteers to evaluate the safety, tolerability and pharmacokinetics (PK) of a device as described herein containing a microtablet as described herein containing 0.5 mg or 0.75 mg ustekinumab (as generally described in Example 3). The study will enroll healthy male and female participants aged 18 to 65, inclusive, at the time of consent, subject to screening for inclusion and exclusion criteria. The study will include three groups:
Group 1 (STELARA®): Participants will be fasted for at least 8 hours. A pre-dose PK sample will be collected and then the participants will be administered a 0.5 mg dose of Stelara® via subcutaneous.
Group 2: Participants will be fasted for at least 8 hours. A pre-dose PK sample will be collected and then the participants will orally ingest a capsule as described herein containing a 0.5 mg dose of ustekinumab, to be swallowed intact with water.
Group 3: Participants will be fasted for at least 8 hours. A pre-dose PK sample will be collected and then the participants will orally ingest a capsule as described herein containing a 0.75 mg dose of ustekinumab, to be swallowed intact with water.

Safety and pK assessments will be made periodically through day 60. The primary endpoints will include the type, incidence, timing, and severity of reported adverse events, and whether they are related to the capsule or the drug. The secondary endpoints will include serum PK parameters, including Cmax, Tmax and area under the serum concentration curve (AUC).

What is claimed is:

1. A method of administering anti-interleukin antibodies to a subject in need thereof, comprising administering to the subject by ingestion an ingestible device containing a payload formed from, or containing, a composition comprising anti-interleukin antibodies, wherein the device is structured to deliver the composition into a gastrointestinal (GI) lumen wall of the subject or into and through a GI lumen wall into a peritoneum or peritoneal cavity of the subject, wherein the anti-interleukin antibodies bind to one or both of human interleukin-23 and human interleukin-12.

2. The method of claim 1, wherein the anti-interleukin antibodies comprise ustekinumab or a biosimilar thereof.

3. The method of claim 1, wherein the anti-interleukin antibodies comprise ustekinumab.

4. The method of claim 1, wherein the device contains a dose of anti-interleukin antibodies of from about 0.25 mg to about 0.75 mg.

5. The method of claim 1, wherein the device contains a dose of anti-interleukin antibodies selected from about 0.5 mg and about 0.75 mg.

6. The method of claim 1, wherein the device contains a dose of anti-interleukin antibodies of from about 3.75 to about 15 mg.

7. The method of claim 1, comprising once daily administration of the device.

8. The method of claim 1, wherein the composition is a solid composition that comprises the anti-interleukin antibodies, a buffering agent, a surfactant, and an antioxidant.

9. The method of claim 1, wherein the composition is a liquid composition.

10. The method of claim 1, wherein the payload is in the form of a solid tissue penetrating member structured to be inserted into a GI lumen wall of the subject and/or be inserted into a peritoneum or peritoneal cavity of the subject after ingestion of the device.

11. The method of claim 10, wherein the composition is filled in a hollow, biodegradable microneedle constituting the solid tissue penetrating member.

12. The method of claim 10, wherein the composition is a shaped mass constituting the solid tissue penetrating member.

13. The method of claim 1, wherein the device comprises multiple payloads of the anti-interleukin antibodies, optionally wherein the device is structured to deliver one or more payloads into the GI lumen wall, peritoneum, or peritoneal cavity of the subject at different times.

14. The method of claim 1, wherein the ingestible device is comprised within a swallowable capsule.

15. The method of claim 1, wherein the subject is suffering from one or more conditions selected from psoriasis, moderate to severe plaque psoriasis, psoriatic arthritis, Crohn's disease, and ulcerative colitis.

16. The method of claim 1, wherein the GI lumen wall is a wall of the stomach.

17. The method of claim 1, wherein the GI lumen wall is a wall of the small intestine.

18. The method of claim 1, wherein the method comprises an induction dosing period following by a maintenance dosing period.

19. The method of claim 18, wherein the subject is an adult patient weighing ≤100 kg or a child patient weighing >60 kg, and
the induction dosing period comprises an induction dosing regimen selected from:
a 45 mg subcutaneous dose at week 0 and week 4;
once daily administration of about 1.5 mg by the ingestible device for a period of time on the order of 4 weeks;
once daily administration of about 12 mg by the ingestible device for a period of time on the order of 4 weeks;
once daily administration of about 9 mg/day by the ingestible device for 7 days beginning at week 0 and again at week 4; and
the maintenance dosing period comprises a maintenance dosing regimen selected from:
a 45 mg subcutaneous dose every 12 weeks;
once daily administration of about 0.5 mg by the ingestible device;
once weekly administration of about 3.75 mg by the ingestible device;
a once monthly short course of daily administration of about 2.5 mg/day by the ingestible device for 6 days;
once quarterly administration of about 15 mg by the ingestible device;
a once quarterly short course of daily administration of about 12 mg/day by the ingestible device for 5 days;
wherein the amounts refer to the dose of anti-interleukin antibody, optionally wherein the anti-interleukin antibody is ustekinumab or a biosimilar thereof,
wherein the method comprises administration of at least one dose of anti-interleukin antibody by the ingestible device.

20. The method of claim 18, wherein the subject is an adult patient weighing >100 kg, and
the induction dosing period comprises an induction dosing regimen selected from:
a 90 mg subcutaneous dose at week 0 and week 4;
once daily administration of about 3.25 mg by the ingestible device for a period of time on the order of 4 weeks;
twice daily administration of about 1.5 mg by the ingestible device for a period of time on the order of 4 weeks;
once daily administration of about 24 mg by the ingestible device for a period of time on the order of 4 weeks;
once daily administration of about 18 mg/day by the ingestible device for 7 days beginning at week 0 and again at week 4; and
the maintenance dosing period comprises a maintenance dosing regimen selected from:
a 90 mg subcutaneous dose every 12 weeks;
once daily administration of about 1 mg by the ingestible device;
twice daily administration of about 0.5 mg by the ingestible device;
once weekly administration of about 7.5 mg by the ingestible device;
a once monthly short course of twice daily administration of about 2.5 mg by the ingestible device for 6 days;
once quarterly administration of about 30 mg by the ingestible device;
a once quarterly short course of daily administration of about 12 mg/day by the ingestible device for 9 days;
wherein the amounts refer to the dose of anti-interleukin antibody, optionally wherein the anti-interleukin antibody is ustekinumab or a biosimilar thereof,
wherein the method comprises administration of at least one dose of anti-interleukin antibody by the ingestible device.
